(19) United States
(12) Patent Application Publication
(10) Pub. No.: US 2025/0127882 A1
(43) Pub. Date: Apr. 24, 2025

(54) NUCLEIC ACID-LIPID NANOPARTICLE AND METHOD USING THE SAME

(71) Applicants: Chiou-Hwa YUH, Rosemead, CA (US); NATIONAL HEALTH RESEARCH INSTITUTES, Miaoli County (TW)

(72) Inventors: Shih-Jen LIU, Miaoli County (TW); Ming-Hsi HUANG, Miaoli County (TW); Hsin-Wei CHEN, Miaoli County (TW); Ching-Len LIAO, Miaoli County (TW)

(21) Appl. No.: 18/836,172

(22) PCT Filed: Mar. 7, 2023
(86) PCT No.: PCT/US2023/014699

§ 371 Date: Aug. 6, 2024

Related U.S. Application Data
(60) Provisional application No. 63/319,028, filed on Mar. 11, 2022.

(51) Int. Cl. A61K 39/215; A61K 9/1271; A61K 9/51; A61K 39/00; A61P 31/14; C12N 7/00

(52) U.S. Cl. CPC A61K 39/215; A61K 9/1271; A61K 9/5123; A61P 31/14; C12N 7/00; A61K 2039/53; A61K 2039/55555; A61K 2039/575; C12N 2770/20034

(57) ABSTRACT

A nucleic acid-lipid nanoparticle is provided, which comprises: a nucleic acid molecule and a lipid mixture. The lipid mixture comprises: an ionizable amino lipid present in an amount of 20 mol % to 60 mol %; a phospholipid present in an amount of 5 mol % to 20 mol %; cholesterol present in an amount of 25 mol % to 60 mol %; and a PEGylated lipid present in an amount of 0.2 mol % to 6 mol %. In addition, methods using the aforesaid nucleic acid-lipid nanoparticle are also provided.

### CROSS-REFERENCE TO RELATED APPLICATIONS

This application claims the benefit of filing date of U.S. Provisional Application Ser. No. 63/319,028, entitled “Methods and components of nucleic acid vaccine” filed Mar. 11, 2022 under 35 USC § 119(e)(1).

### BACKGROUND OF THE INVENTION

### Field

The present invention relates to a nucleic acid-lipid nanoparticle and methods using the same. More specifically, the present invention relates to a nucleic acid-lipid nanoparticle as a nucleic acid vaccine and methods using the same.

### Description of Related Art

Currently, a series of vaccine candidates against emerging viral pandemic coronavirus disease 2019 (COVID-19) are in development around the world, and several of these candidate vaccines are either authorized for emergency use or approved for used in humans. Among these, the nucleic acid (NA)-based vaccines contain a section of genetic material of severe acute respiratory syndrome coronavirus 2 (SARS-CoV-2) that gives rise to a person's own cells to make parts of the targeted viral particles against which immunity is generated, allowing rapid, scalable, and cost-effective production. Despite messenger RNA (mRNA) has received the major interest and became a new era in vaccinology, RNA molecules are of low stability and require protection from enzymatic digestion to achieve successful targeting delivery. From COVID-19 vaccine experience, the successful advancement of lipid nanoparticle (LNP) technology toward more sophisticated has highlighted the gap between the advantages of mRNA carrier platforms and the availability of manufacturing processes to scale up their production. However, the requirement of ultra-cold transportation limits the feasibility for communities lacking cold chain facilities. Different from mRNA vaccines, DNA molecules possess high stability and do not require ultra-cold storage and distribution; however, efficacious DNA vaccine delivery as well as protective immunity against SARS-CoV-2 infection required the aid of either a viral vector or electroporation into muscle or skin of the host.

The formation of nucleic acid-LNP is a self-assembling process between two miscible fluids, a buffer solution of a nucleic acid molecule and a water-miscible solution (ethanol) comprising the lipids mixture, the latter commonly contains four hybrid components dissolved in ethanol:cholesterol (for cell transfection), phospholipid (a helper lipid for particle structure), an ionizable cationic lipidoid (for cellular uptake and endosomal escape of nucleic acid) and a PEGylated lipidoid (for LNP stability/circulation). Each component makes contribution to the structural stability and functional activity. Both cholesterol and phospholipids are building block components embedded in the lipid bilayer of the cell membrane. Most studies have been published on the general properties and design features of ionizable cationic lipidoids, in more particular on the structural function of the designed lipids and the identification of mRNA vaccine-induced adaptive immune responses those are associated with the expression kinetics and the endosomal escape activities. On the other hand, PEGylated lipidoids act as surfactants that stand out of the surface of LNP to reduce surface free energy between the lipophilic core and aqueous phase as well as nonspecific binding to proteins by steric repulsion.

Since the LNP carrier technology is a potential tool for the development of effective nucleic acid vaccination, it is desirable to provide a novel nucleic acid-lipid nanoparticle to extend the field of using LNP systems for delivering different nucleic acid molecules.

### SUMMARY OF THE INVENTION

The present invention provides a nucleic acid-lipid nanoparticle, which comprises: a nucleic acid molecule and a lipid mixture. The lipid mixture comprises: an ionizable amino lipid present in an amount of 20 mol % to 60 mol %; a phospholipid present in an amount of 5 mol % to 20 mol %; cholesterol present in an amount of 25 mol % to 60 mol %; and a PEGylated lipid present in an amount of 0.2 mol % to 6 mol %.

The present invention also provides a method for preventing or ameliorating a coronavirus infection, comprising administering the aforesaid nucleic acid-lipid nanoparticle to a subject in need thereof.

The present invention further provides a method for inducing an immune response against a coronavirus in a subject, comprising administering the aforesaid nucleic acid-lipid nanoparticle to a subject in need thereof.

The present invention further provides a method for administering a booster dose to a vaccinated subject, comprising administering the aforesaid nucleic acid-lipid nanoparticle to a subject who was previously vaccinated against coronavirus.

Other novel features of the disclosure will become more apparent from the following detailed description when taken in conjunction with the accompanying drawings.

### BRIEF DESCRIPTION OF DRAWINGS

FIG. 1A to FIG. 1C are diagrams respectively showing the formulation efficiency, diameter, particle size distribution and surface charge of the mRNA-LNP with different PLA-PEG contents.

FIG. 2A to FIG. 2C are diagrams respectively showing the formulation efficiency, diameter, particle size distribution and surface charge of the mRNA-LNP with different DSPC contents.

FIG. 3A to FIG. 3C are diagrams respectively showing the formulation efficiency, diameter, particle size distribution and surface charge of the mRNA-LNP with different PLA-PEG to DSPC (P/H) ratios.

FIG. 4A to FIG. 4C are diagrams respectively showing the formulation efficiency, diameter, particle size distribution and surface charge of the mRNA-LNP with different SM-102 contents.

FIG. 5A to FIG. 5C are diagrams respectively showing the formulation efficiency, diameter, particle size distribution and surface charge of the mRNA-LNP with different mRNA contents.

FIG. 6 shows the photos of 293T cells treated with mRNA-encoding eGFP in serum-free growth medium or within mRNA-LNP formulations (mRNA-LNP-FM, mRNA-LNP-FB, mRNA-LNP-F2) or in the presence of conventional transfection reagent, lipofectamine.

FIG. 7A and FIG. 7B are diagrams showing the stability of mRNA-LNPs when 1×10⁵ of 293T cells treated with 1.0 μg mRNA-luciferase within mRNA-LNP formulations (mRNA-LNP-F2 and mRNA-LNP-FM) in triplicate cultures for 48 hrs.

FIG. 8A and FIG. 8B are diagrams showing the stability of mRNA-LNPs when 2×10⁵ of 293T cells treated with 1.0 μg mRNA-luciferase within mRNA-LNP formulations (mRNA-LNP-F2 and mRNA-LNP-FM) in triplicate cultures for 48 hrs.

FIG. 8C and FIG. 8D are diagrams showing the stability of mRNA-LNPs when 1×10⁵ of 293T cells treated with 1.0 μg mRNA-luciferase within mRNA-LNP formulations (mRNA-LNP-F2 and mRNA-LNP-FM) in triplicate cultures for 48 hrs.

FIG. 9A to 9C are diagrams showing the results of in vivo bioluminescence of mRNA-LNPs.

FIG. 10 shows the photos of 293T cells treated with DNA-GFP using DNA Turbojet Transfection Reagent or LNP-encapsulated.

FIG. 11A to FIG. 11C are diagrams respectively showing the formulation efficiency, diameter, particle size distribution and surface charge of the DNA-LNP with different PLA-PEG contents.

FIG. 12A to FIG. 12C are diagrams respectively showing the formulation efficiency, diameter, particle size distribution and surface charge of the DNA-LNP with different DSPC contents.

FIG. 13A to FIG. 13C are diagrams respectively showing the formulation efficiency, diameter, particle size distribution and surface charge of the DNA-LNP with different PLA-PEG to DSPC (P/H) ratios.

FIG. 14A to FIG. 14C are diagrams respectively showing the formulation efficiency, diameter, particle size distribution and surface charge of the DNA-LNP with different SM-102 contents.

FIG. 15A to FIG. 15C are diagrams respectively showing the formulation efficiency, diameter, particle size distribution and surface charge of the DNA-LNP with different DNA contents.

FIG. 16A to FIG. 16C are diagrams respectively showing the formulation efficiency, diameter and luciferase activity when the DNA-LNP-F2 were stored at different temperatures.

FIG. 17 presents the photos showing the in vivo expression of DNA-LNP measured by in vivo luminescence.

FIG. 18A to FIG. 18E are diagrams respectively showing the particle size, particle size distribution, surface charge, formulation efficiency and transfection results of different DNA-LNP formulations.

FIG. 19A to FIG. 19F are diagrams respectively showing the encapsulation efficiency, particle size, 293T luciferase, recovery rate, particle size distribution and RAW264.7 luciferase of different DNA-LNP formulations.

FIG. 20A is a schematic diagram of vaccination schedule of Example 13.

FIG. 20B is a diagram showing the results of neutralizing antibody titer against TW04 strain.

FIG. 20C to FIG. 20E are diagrams respectively showing the results of the anti-trimeric spike protein IgG titer at week 3, 5 and 7.

FIG. 21A is a schematic diagram of vaccination schedule of Example 14.

FIG. 21B and FIG. 21C are diagrams respectively showing the results of the anti-trimeric spike protein IgG titer at week 2 and 3.

FIG. 22A is a schematic diagram of vaccination schedule of Example 15.

FIG. 22B is a diagram showing the virus neutralizing titre at week 6.

FIG. 22C and FIG. 22D are diagrams showing the virus titre at 3- and 6-days post-infection (d.p.i.).

FIG. 22E is a diagram showing the evaluation results of histopathological examination of tissue sections.

FIG. 22F is a diagram of the body weight change (%) after virus challenge.

FIG. 23 is a diagram showing the virus neutralizing titre against SARS-CoV-2 Omicron variant when using the DNA-LNP formulation as a booster dose.

### DETAILED DESCRIPTION OF THE INVENTION

The following embodiments when read with the accompanying drawings are made to clearly exhibit the above-mentioned and other technical contents, features and/or effects of the present disclosure. Through the exposition by means of the specific embodiments, people would further understand the technical means and effects the present disclosure adopts to achieve the above-indicated objectives. Moreover, as the contents disclosed herein should be readily understood and can be implemented by a person skilled in the art, all equivalent changes or modifications which do not depart from the concept of the present disclosure should be encompassed by the appended claims.

Furthermore, the ordinals recited in the specification and the claims such as “first”, “second” and so on are intended only to describe the elements claimed and imply or represent neither that the claimed elements have any proceeding ordinals, nor that sequence between one claimed element and another claimed element or between steps of a manufacturing method. The use of these ordinals is merely to differentiate one claimed element having a certain designation from another claimed element having the same designation.

Moreover, in the present specification, a value may be interpreted to cover a range within ±10% of the value, and in particular, a range within ±5% of the value, except otherwise specified; a range may be interpreted to be composed of a plurality of subranges defined by a smaller endpoint, a smaller quartile, a median, a greater quartile, and a greater endpoint, except otherwise specified.

“An effective amount” refers to the amount of the active ingredient which is required to confer the desired effect on the subject. Effective amounts vary, as recognized by those skilled in the art, depending on route of administration, excipient usage, and the possibility of co-usage with other therapeutic treatments such as use of other active agents.

The present invention provides a nucleic acid-lipid nanoparticle, which comprises: a nucleic acid molecule and a lipid mixture. The lipid mixture comprises: an ionizable amino lipid present in an amount of 20 mol % to 60 mol %; a phospholipid present in an amount of 5 mol % to 20 mol %; cholesterol present in an amount of 25 mol % to 60 mol %; and a PEGylated lipid present in an amount of 0.2 mol % to 6 mol %.

In one embodiment, the nucleic acid molecule may comprise a messenger RNA (mRNA) or a DNA encoding a part of a virus. In one embodiment, the nucleic acid molecule may comprise a mRNA or a DNA encoding a part of a coronavirus. In one embodiment, the nucleic acid molecule may comprise a mRNA or a DNA encoding a part of SARS-CoV-2 virus. In one embodiment, the nucleic acid molecule comprises a mRNA or a DNA encoding a trimeric spike protein of SARS-CoV-2 virus. In one embodiment, the nucleic acid molecule may comprise a mRNA encoding a trimeric spike protein of SARS-CoV-2 virus. In another embodiment, the nucleic acid molecule may comprise a DNA encoding a trimeric spike protein of SARS-CoV-2 virus.

In one embodiment, the nucleic acid molecule may comprise a messenger RNA (mRNA) encoding luciferase.

In one embodiment, the amount of the ionizable amino lipid in the lipid mixture may range from 20 mol % to 60 mol %, for example, from 20 mol % to 55 mol % or from 33 mol % to 52 mol %.

In one embodiment, the amount of the phospholipid in the lipid mixture may range from 5 mol % to 20 mol %, for example, from 5 mol % to 15 mol % or from 8 mol % to 13 mol %.

In one embodiment, the amount of the cholesterol in the lipid mixture may range from 25 mol % to 60 mol %, for example, from 30 mol % to 55 mol % or from 35 mol % to 50 mol %.

In one embodiment, the amount of the PEGylated lipid in the lipid mixture may range from 0.2 mol % to 6 mol %, for example, from 0.3 mol % to 5 mol % or from 0.5 mol % to 4 mol %.

In one embodiment, the ionizable amino lipid may be selected from the group consisting of heptadecan-9-yl 8-((2-hydroxyethyl) (6-oxo-6-(undecyloxy) hexyl)amino)octanoate (SM-102), (6Z,9Z,28Z,31Z)-heptatriaconta-6,9,28,31-tetraen-19-yl-4-(dimethylamino)butanoate (DLin-MC3-DMA), ((4-hydroxybutyl)azanediyl)bis(hexane-6,1-diyl)bis(2-hexyldecanoate) (ALC-0315) and a combination thereof. In one embodiment, the ionizable amino lipid may comprise SM-102. In another embodiment, the ionizable amino lipid may comprise DLin-MC3-DMA. In further another embodiment, the ionizable amino lipid may comprise ALC-0315.

In one embodiment, the phospholipid as a helper lipid may be selected from the group consisting of dioleoylphosphatidyl ethanolamine (DOPE), dimyristoylphosphatidyl choline (DMPC), 1,2-distearoyl-sn-glycero-3-phosphocholine (DSPC, also called as distearoylphosphatidyl choline), dimyristoylphosphatidyl glycerol (DMPG), dipalmitoyl phosphatidylcholine (DPPC), phosphatidylcholine (PC) and a combination thereof. In one embodiment, the phospholipid may comprise DSPC.

In one embodiment, the PEGylated lipid may comprise 1,2-dimyristoyl-rac-glycero-3-methoxypolyethylene glycol (DMG-PEG), a poly(lactic acid)-PEG (PLA-PEG) copolymer, a poly(lactide-co-F-caprolactone)-block-poly(ethylene glycol) (PLACL-PEG) copolymer or a combination thereof.

In one embodiment, the PEGylated lipid may comprise DMG-PEG. In one embodiment, PEG in the DMG-PEG may have an average molecular weight of 1,500 Daltons to 3,000 Daltons. In one embodiment, PEG in the DMG-PEG may have an average molecular weight of about 2,000 Daltons (for example, DMG-PEG 2000).

In one embodiment, the PEGylated lipid may comprise a PLA-PEG copolymer. In one embodiment, the PLA-PEG copolymer may be a copolymer having a first block of PLA and a second block of PEG. In another embodiment, the PLA-PEG copolymer may be a copolymer having a first block of PLA, a second block of PEG and a third block of PLA, and the second block of PEG is arranged between the first block of PLA and the third block of PLA.

In one embodiment, the first block of PLA or the third block of PLA of the PLA-PEG copolymer may have an average molecular weight of 200 Daltons to 600 Daltons, for example, 200 Daltons to 550 Daltons or 250 Daltons to 500 Daltons. In one embodiment, the first block of PLA and the third block of PLA of the PLA-PEG copolymer may respectively have an average molecular weight of about 250 Daltons. In another embodiment, the first block of PLA and the third block of PLA may respectively have an average molecular weight of about 500 Daltons.

In one embodiment, the second block of PEG of the PLA-PEG copolymer may have an average molecular weight of 1,500 Daltons to 6,000 Daltons, for example, 1,600 Daltons to 5,500 Daltons, 1,800 Daltons to 5,000 Daltons or 2,000 Daltons to 4,600 Daltons. In one embodiment, the second block of PEG of the PLA-PEG copolymer may have an average molecular weight of about 2,000 Daltons. In another embodiment, the second block of PEG of the PLA-PEG copolymer may have an average molecular weight of about 4,600 Daltons.

In one embodiment, the PEGylated lipid may comprise a PLACL-PEG copolymer. In one embodiment, the PEGylated lipid may comprise a copolymer having a first block of poly(lactide-co-ε-caprolactone) and a second block of PEG.

In one embodiment, the first block of poly(lactide-co-ε-caprolactone) of the PLACL-PEG copolymer may have an average molecular weight of 1,500 Daltons to 2,500 Daltons, for example, 1,700 Daltons to 2,300 Daltons. In one embodiment, the first block of poly(lactide-co-ε-caprolactone) of the PLACL-PEG copolymer may have an average molecular weight of about 2,000 Daltons.

In one embodiment, the second block of PEG of the PLACL-PEG copolymer may have an average molecular weight of 4,000 Daltons to 6,000 Daltons, for example, 4,500 Daltons to 5,500 Daltons. In one embodiment, the second block of PEG of the PLACL-PEG copolymer may have an average molecular weight of about 5,000 Daltons.

In one embodiment, a weight ratio of the lipid mixture (total lipid) to the nucleic acid molecule may range from 50:1 to 5:1, for example, 40:1 to 5:1, 30:1 to 5:1, 20:1 to 5:1, 15:1 to 5:1 or 12:1 to 6:1.

In one embodiment, the nucleic acid-lipid nanoparticle may have a median diameter ranging from 80 nm to 240 nm, for example, 90 nm to 240 nm, 90 nm to 230 nm, 100 nm to 230 nm or 100 nm to 225. However, the present invention is not limited thereto, and the median diameter of the nucleic acid-lipid nanoparticle may be varied according to the used nucleic acid molecule and the used lipid mixture.

The present invention provides a novel nucleic acid-lipid nanoparticle, which can be used to prepare a vaccine composition that targets virus, for example, SARS-CoV-2 virus including different variants of SARS-CoV-2 virus.

The present invention also provides a method for preventing or ameliorating a coronavirus infection, and the method comprises administering the aforesaid nucleic acid-lipid nanoparticle to a subject in need thereof.

The present invention also provides a use of the aforesaid nucleic acid-lipid nanoparticle for manufacturing a vaccine composition for preventing or ameliorating a coronavirus infection.

The present invention also provides a method for inducing an immune response against a coronavirus in a subject, and the method comprises administering the aforesaid nucleic acid-lipid nanoparticle to a subject in need thereof.

The present invention also provides a use of the aforesaid nucleic acid-lipid nanoparticle for manufacturing a vaccine composition for inducing an immune response against a coronavirus in a subject.

The present invention also provides a method for administering a booster dose to a vaccinated subject, and the method comprises administering the aforesaid nucleic acid-lipid nanoparticle to a subject who was previously vaccinated against coronavirus.

The present invention also provides a use of the aforesaid nucleic acid-lipid nanoparticle for manufacturing a vaccine composition as a booster dose to a vaccinated subject.

In the present invention, the coronavirus in any of the aforesaid methods or the aforesaid uses may be SARS-CoV-2 virus.

### Materials and Methods

### Plasmid Construction and Characterization

The DNA molecules used in the following examples were the DNA sequences encoding full-length SARS-CoV-2 spike genes (GenBank accession number: MN908947), which were optimized for human codon usage and synthesized by GenScript Biotech, New Jersey. The plasmid DNA encoding SARS-CoV-2 Trimeric-Spike Omicron as previously described. In cell culture study and in vivo distribution assessment, plasmid DNA encoding enhanced green fluorescent protein (eGFP) or green click beetle luciferase protein (CBGr99) were used as reporter genes for monitoring the transfection/expression of the vaccine. These genes were subcloned into the clinically used vector pVAX1 with the Kozak sequence incorporated at the 5′ end of the genes as previously described. eGFP is expressed by a pcDNA3.1 backbone plasmid vector. The plasmids were transformed into E. coli DH50α cells (ECOS™101, Yeastern Biotech co., Taiwan) for plasmid amplification. Plasmids were extracted and purified using an endotoxin-free Qiagen column system (EndoFree Plasmid Mega Kit, cat #12381, QIAGEN, Germany).

mRNA In Vitro Transcription

The vaccine compositions used in this invention was an in vitro-transcribed (IVT) mRNA contains five structural elements: a 5′ cap, a 5′ untranslated region (UTR), an open reading frame (ORF) that encodes the antigen, a 3′ UTR and a poly(A) tail. The ORF contains a nucleic acid sequence encoding the SARS-CoV-2 Spike protein (see Liao H C, Wu W L, Chiang C Y, et al. Low-Dose SARS-CoV-2 S-Trimer with an Emulsion Adjuvant Induced Th1-Biased Protective Immunity, Int J Mol Sci. 2022; 23(9):4902.) being designed to contain a nonfunctional furin cleavage site (R682G, R683S, R685S) and two stabilizing prolines (K986P, V987P) in the hinge loop. Additionally, the transmembrane domain and the C-terminal intracellular tail were removed (S-2P) or replaced by a trimerization domain IZN4 (S-Trimer) as model antigen to generate protective immunity against virus infection. ORF contains a nucleic acid sequence encoding green click beetle luciferase protein (CBGr99) as model signal for transfection/expression in cell culture system and in vivo distribution. In addition, the mRNA-encoding eGFP was purchased from a local company (BIOTOOLS Co., Ltd, New Taipei, Taiwan). The DNA template for mRNA in vitro transcription was linearized pT7TSomi plasmid with restriction enzyme, BamH I and Xbal I (Fermentas). After DNA linearization, the DNA template was resolved in H₂O to 1 μg/μL and 1 μg for a 40 μL transcription reaction. The N1-mythel-pseudoUTP and CleanCap AG 3′Ome (Trilink) were used in this reaction.

### In Vitro Transfection

24-well plates were seeded with 293T cells or RAW264.7 cells in 10% FBS (Cat. #10437-028, Gibco, Australia) DMEM (Cat. #SH30243.02, Hyclone, Utah, USA) medium at a total volume of 1 ml per well. At 24 hrs post-seeding, cells were transfected in triplicate in the presence of 1 μg of nucleic acid-LNP. Cells transfected with commercial transfection reagent, TurboFect™ (Cat. #R0531, Thermo-Fisher Scientific, Lithuania), was used as a positive control. Complexes were prepared by pre-incubation of 1 μg DNA and 2 μl TurboFect™ in 100 μl serum-free DMEM at room temperature for 15 min. Plates were then incubated for 3 days at 37° C. and 5% CO₂ in air. Two reporter genes were selected as indicators for transfection efficiency:enhanced green fluorescent protein (eGFP) and green click beetle luciferase protein (CBGr99).

### In Vitro Imaging

After 72 hrs' transfection by eGFP nucleic acid-LNP, 5×10⁴ transfected 293T cells were cultured in 24-well plates (Cat. #142475, Nunc, Thermo-Fisher Scientific, China) in 1 ml 10% FBS DMEM, images were taken with a fluorescent microscope (Olympus IX73 inverted microscope, Tokyo, Japan). Cell nucleuses were stained with Hoechst 33342 dye (Thermo-Fisher Scientific, Rockford, IL, USA) before imaging.

### Ethics Statements

BALB/c mice and Syrian hamsters were obtained from the National Laboratory Animal Breeding and Research Centre (Taipei, Taiwan). All animals were housed at the Animal Centre of the National Health Research Institutes (NHRI) and maintained in accordance with institutional animal care protocols (Protocol No: NHRI-IACUC-109077-A).

### In Vivo Luminescence in Mice

Transfection of DNA molecules and expression of luciferase protein in mice was measured by using in vivo luminescence. Mice were inoculated with predetermined amounts of the nucleic acid-LNP (10 μg per mouse) via intramuscular (i.m.) route. Day 1, 2, 3, 4, 7, 10, 14, 21, 28 after administrations, animals were injected intraperitoneally (i.p.) at a dose of 150 mg per kg of mouse body weight with luciferin/PBS (15 mg ml⁻¹; Cat. #ab143655, Abcam, Cambridge, UK) and waited for the reaction to take place. Luminescence signals were collected by IVIS Spectrum instrument (Perkin Elmer, Waltham, MA), and the luminescence signals in regions of interest (ROIs) were quantified using the imaging software “Living Image”.

### Example 1—Preparation and Characterization of mRNA-LNPs

mRNA-LNP formulations were elaborated using microfluidic approaches. The process is consistent and reproducible, such that the LNP products can be easily scaled up from lab scale to GMP scale. Briefly, lipids (ionizable cationic lipid:cholesterol:helper lipid:PEGylated lipid) were dissolved in ethanol at predetermined molar ratios. The lipid mixture was combined with a mRNA-containing buffer solution at a volume ratio of 1/3 (ethanol/aqueous) using a microfluidic mixer (Precision Nanosystems, Vancouver, BC, Canada). Formulations were then concentrated and stored in the TRIS (pH 7.4) at room temperature or 4° C. until use. LNP characterization was performed, including particle size distribution using laser light scattering techniques, RNA determination and encapsulation efficiency (EE) using fluorescence detection method (RiboGreen™ RNA Reagent and Quantitation Kit). The results are listed in the following Table 1.

TABLE 1

| | Characteristics | | Molar ratio (mol %) of lipids | | | | Types of |
|---|---|---|---|---|---|---|---|
| | Size | EE | | | | PEGylated | PEGylated |
| Acronym | (nm) | (%) | SM-102 | Cholesterol | DSPC | lipid | lipid |
| mRNA-LNP-FM | 103 ± 1 | 71 | 50 | 38.5 | 10 | 1.5 | DMG- |
| | | | (50 mol %) | (38.5 mol %) | (10 mol %) | (1.5 mol %) | PEG 2000 |
| mRNA-LNP-F0 | 209 ± 12 | 51 | 50 | 38.5 | 10 | — | — |
| | | | (50.8 mol %) | (39.1 mol %) | (10.1 mol %) | | |
| mRNA-LNP-F1 | 191 ± 2 | 46 | 50 | 38.5 | 10 | 0.5 | PLA 500- |
| | | | (50.5 mol %) | (38.9 mol %) | (10.1 mol %) | (0.5 mol %) | PEG 2000 |
| mRNA-LNP-F2 | 162 ± 4 | 60 | 50 | 38.5 | 10 | 1.5 | PLA 500- |
| | | | (50 mol %) | (38.5 mol %) | (10 mol %) | (1.5 mol %) | PEG 2000 |
| mRNA-LNP-F3 | 225 ± 8 | 36 | 50 | 38.5 | 10 | 3.0 | PLA 500- |
| | | | (49.3 mol %) | (37.9 mol %) | (9.9 mol %) | (2.9 mol %) | PEG 2000 |

The highly reproducible mRNA-lipid nanoparticle can be prepared by a microfluidic mixer (Precision Nanosystems, Vancouver, BC, Canada). Lipids mixture with molar ratios of 50:38.5:10:1.5 (SM-102:cholesterol:DSPC:DMG-PEG) was denoted as mRNA-LNP-FM. The mRNA-LNP-FM has high encapsulation efficiency (71%) obtained from the fluorescent dye detection, and the data of the particle size detected by the laser light scattering technique shows that uniform and solid nanoparticles (103 nm) can be prepared using this system. When the PEGylated lipid in the LNP was removed, the lipid nanoparticles (mRNA-LNP-FO) were easily aggregated, and the particle size was increased (209 nm) and the encapsulation efficiency of mRNA was decreased (51%). Supplements of appropriate amounts of PLA-PEG were facilitated to form highly encapsulated and stable mRNA-lipid nanoparticles (mRNA-LNP-F1, mRNA-LNP-F2 and mRNA-LNP-F3).

### Example 2—Parameter Tuning for the Preparation of mRNA-LNPs

The methods for preparing the mRNA-LNPs are similar to those described in Example 1 and are not repeated again.

TABLE 2

| | | Molar ratio of lipids (with fixed | | | |
|---|---|---|---|---|---|
| | | cholesterol content of 516 μg) | | | |
| | mRNA | | | | PLA |
| | content | | | | 500-PEG |
| Acronym | (μg) | Cholesterol | DSPC | SM-102 | 2000 |
| PLA-PEG-0 | 100 | 38.5 | 10.0 | 50 | 0.0 |
| PLA-PEG-0.5 | 100 | 38.5 | 10.0 | 50 | 0.5 |
| PLA-PEG-1.0 | 100 | 38.5 | 10.0 | 50 | 1.0 |
| PLA-PEG-1.5 | 100 | 38.5 | 10.0 | 50 | 1.5 |
| PLA-PEG-2.0 | 100 | 38.5 | 10.0 | 50 | 2.0 |
| PLA-PEG-3.0 | 100 | 38.5 | 10.0 | 50 | 3.0 |
| DSPC-0 | 100 | 38.5 | 0.0 | 50 | 1.5 |
| DSPC-5 | 100 | 38.5 | 5.0 | 50 | 1.5 |
| DSPC-10 | 100 | 38.5 | 10.0 | 50 | 1.5 |
| DSPC-20 | 100 | 38.5 | 20.0 | 50 | 1.5 |
| P/H-2.5/9 | 100 | 38.5 | 9.0 | 50 | 2.5 |
| P/H-2/9.5 | 100 | 38.5 | 9.5 | 50 | 2.0 |
| P/H-1.5/10 | 100 | 38.5 | 10.0 | 50 | 1.5 |
| P/H-1/10.5 | 100 | 38.5 | 10.5 | 50 | 1.0 |
| P/H-0.5/11 | 100 | 38.5 | 11.0 | 50 | 0.5 |
| SM-102-20 | 100 | 38.5 | 10.0 | 20 | 1.5 |
| SM-102-30 | 100 | 38.5 | 10.0 | 30 | 1.5 |
| SM-102-40 | 100 | 38.5 | 10.0 | 40 | 1.5 |
| SM-102-50 | 100 | 38.5 | 10.0 | 50 | 1.5 |
| SM-102-60 | 100 | 38.5 | 10.0 | 60 | 1.5 |
| mRNA-50 | 50 | 38.5 | 10.0 | 50 | 1.5 |
| mRNA-66 | 66 | 38.5 | 10.0 | 50 | 1.5 |
| mRNA-100 | 100 | 38.5 | 10.0 | 50 | 1.5 |
| mRNA-200 | 200 | 38.5 | 10.0 | 50 | 1.5 |

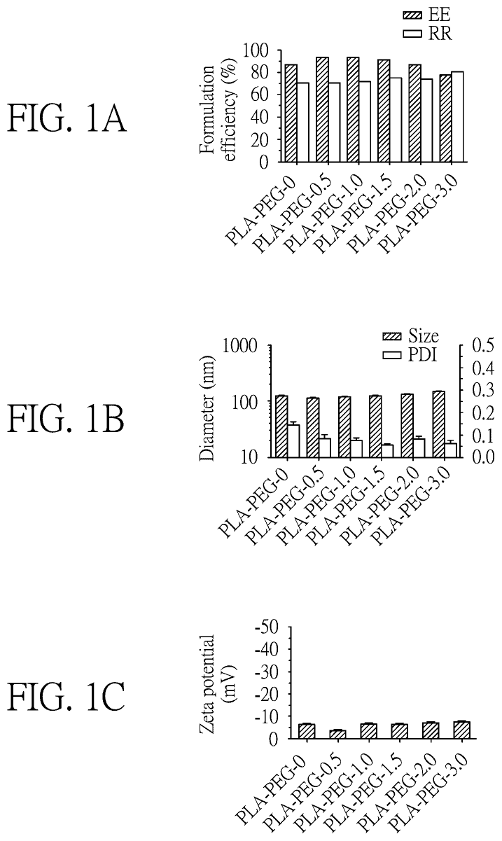

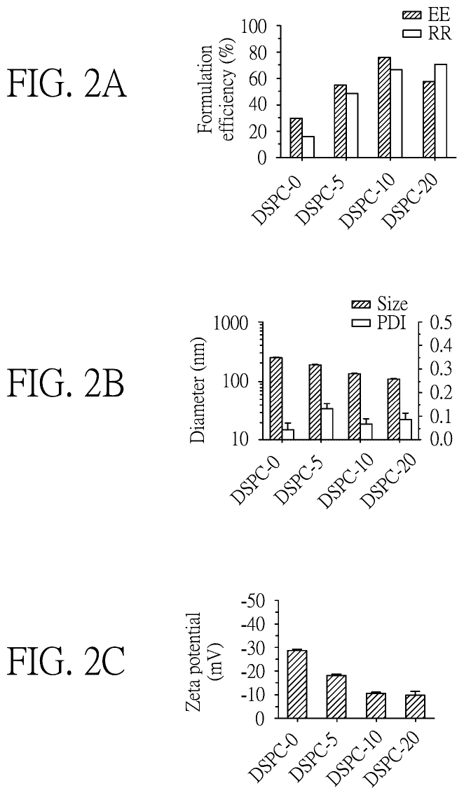

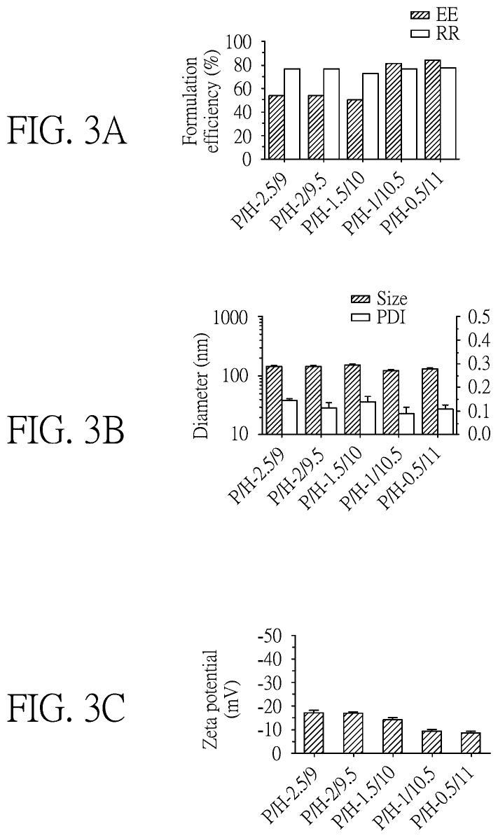

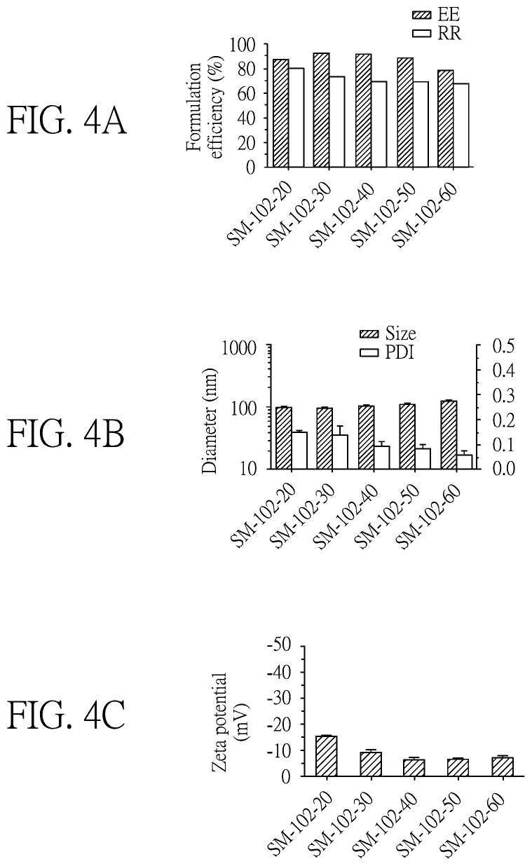

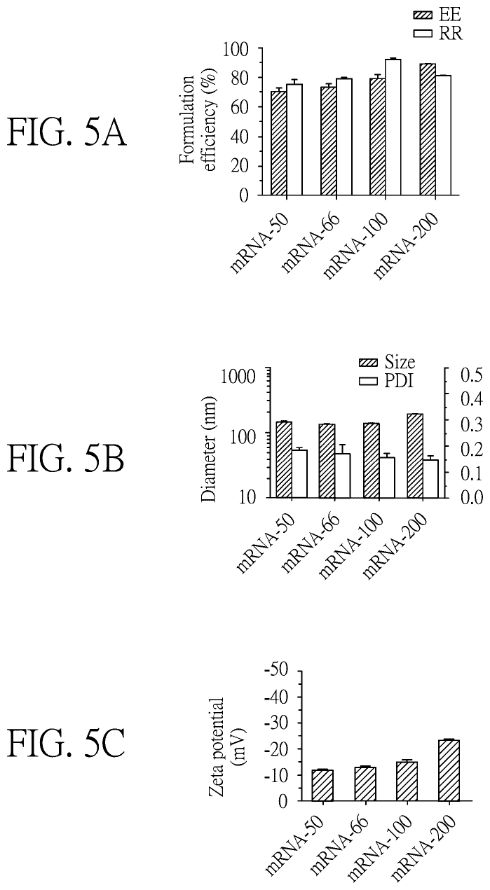

The microfluidic mixer (Precision Nanosystems, Vancouver, BC, Canada) was used to prepare mRNA-LNP-F2, and the contents of the lipid mixture of the mnRNA-LNP-F2 were adjusted, as shown in Table 2. As shown in FIG. 1A to FIG. 5C, it was found that PEGylated lipid (PLA-PEG) mainly affects the particle size distribution of lipid nanoparticles. Other parameters, such as the contents of the helper lipid (DSPC), the ratio of PEGylated lipid to helper lipid (P/H), and the contents of the ionizable cationic lipid (SM-102) all affect encapsulation efficiency (EL) and recovery rate (RR), and also affect the particle size, distribution and surface charge of the lipid nanoparticles. The mRNA content affects the encapsulation efficiency (EE) and recovery rate (RR), as well as the surface charge.

### Example 3—Transfection of mRNA-LNPs

5×10⁴ of 293T cells were treated with 1 μg/ml mRNA-encoding eGFP (Enhanced green fluorescent protein), either in serum-free growth medium or within mRNA-LNP formulations (mRNA-LNP-FM, mRNA-LNP-FB, mRNA-LNP-F2) or in the presence of conventional transfection reagent, lipofectamine. Lipids mixture with molar ratios of 46.3:42.7:9.4:1.6 (ALC-0315:cholesterol:DSPC:ALC-0159) was denoted as mRNA-LNP-FB. Observation of protein expression by fluorescent microscopy and Hoechst staining for cell nuclei after 48 hrs.

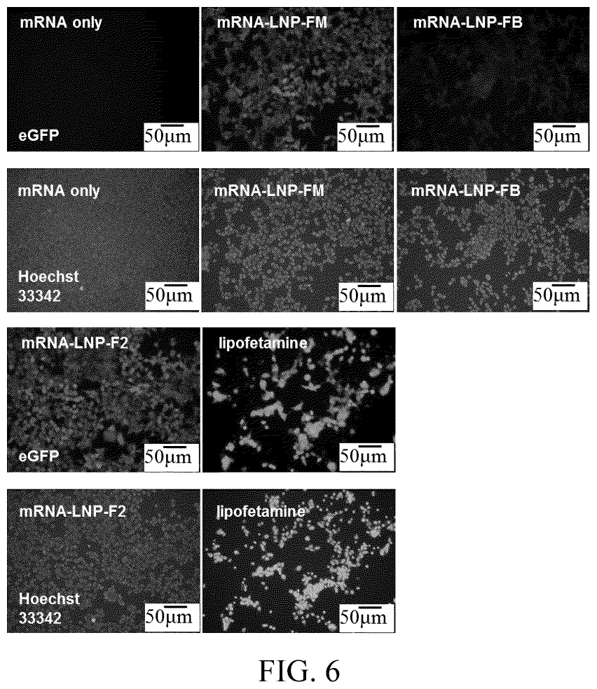

As shown in FIG. 6, when the mRNA-encoding eGFP was used to prepare the lipid mixture of mRNA-LNP-F2, the protein expression is no less than the lipid mixture of mRNA-LNP-FM or mRNA-LNP-FB. In addition, the polymer components used in the present platform are conveniently obtained, which helps to link up with GMP level product manufacturing.

### Example 4—Stability of mRNA-LNPs

Stability monitoring of the mRNA-LNP formulations was performed at room temperature and 4° C. At week 4, the specimens were withdrawn and tested for the transfection of mRNA. 1×10⁵ of 293T cells treated with 1.0 μg mRNA-luciferase within mRNA-LNP formulations (mRNA-LNP-F2 and mRNA-LNP-FM) in triplicate cultures for 48 hrs.

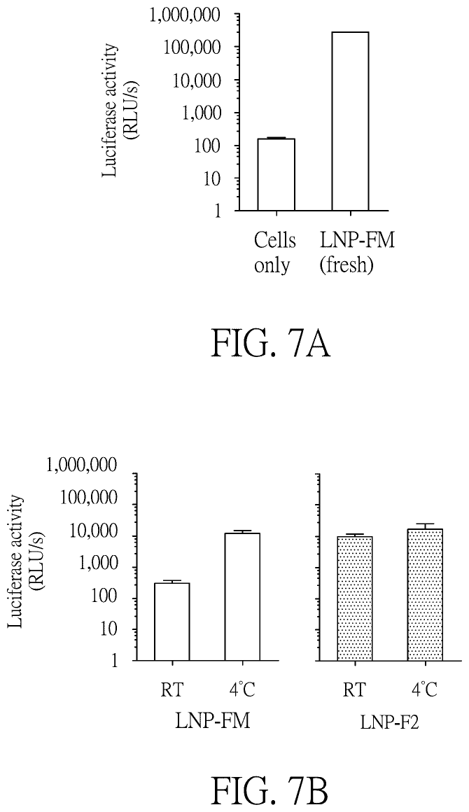

As shown in FIG. 7A and FIG. 7B, according to the luminescence signals of the lipid nanoparticle formulation mRNA-LNP-FM with luciferase in 293T cells, the protein expression was significantly decreased after the mRNA-LNP-FM was stored at room temperature and 4° C. for 4 weeks. On the other hand, the protein expression of mRNA-LNP-F2 was not affected by the temperature after storing at room temperature and 4° C. for 4 weeks.

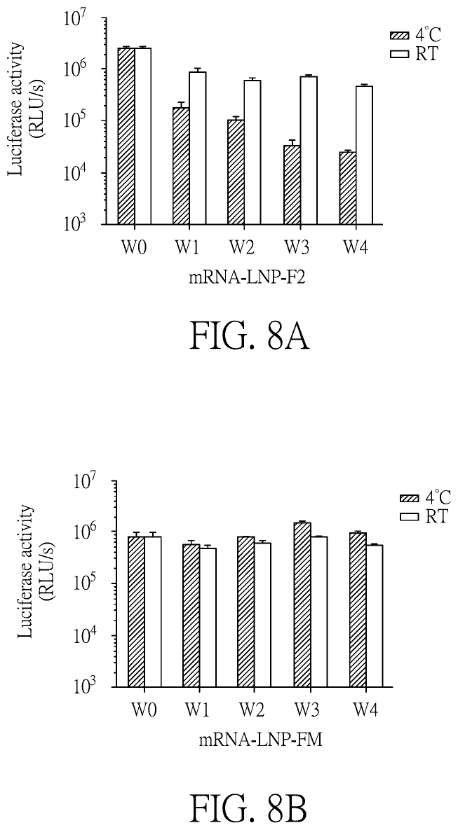

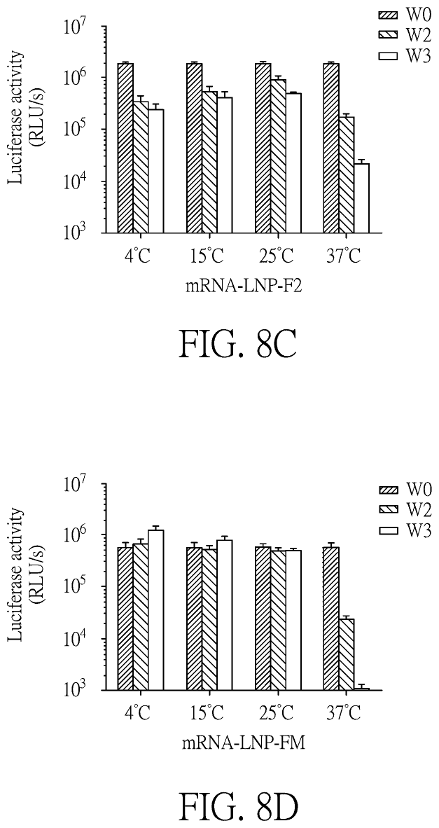

The pre-packed equal volume samples were stored at different temperatures and time, and the luminescence signal produced by the lipid nanoparticle formula with luciferase in 293T cells were observed. As shown in FIG. 8A to 8D, mRNA-LNP-FM is stable when stored at 4° C. or at room temperature (15° C., 25° C.), and its protein expression decreases significantly when stored at 37° C. In addition to being stored at 4° C., mRNA-LNP-F2 has better protein expression ability than mRNA-LNP-FM when stored above room temperature.

### Example 5—In Vivo Bioluminescence of mRNA-LNPs

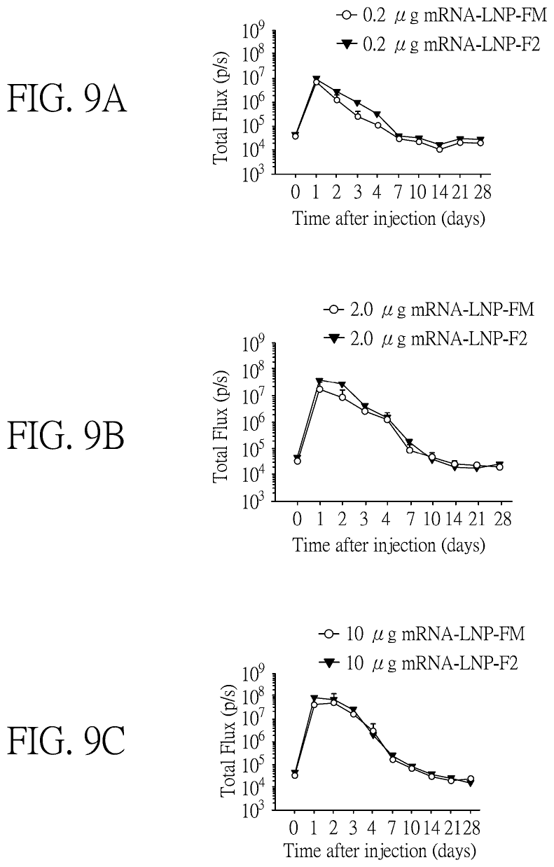

Female BALB/c mice were inoculated with predetermined amounts of the incubated mRNA-LNP-FM or mRNA-LNP-F2 via intramuscular (i.m.) route. Day 1, 2, 3, 4, 7, 10, 14, 21, 28 after administrations, animals were injected intraperitoneally (i.p.) with luciferase substrate, and waited 11 minutes for reaction. Fluorescence signals were recorded by IVIS Spectrum instrument, and the fluorescence signals in regions of interest (ROIs) were quantified using Living Image. The results are shown in FIG. 9A to FIG. 9C.

The mice treated with mRNA-luciferase in mRNA-LNP-FM formulation and in the lipid mixture of the present invention (mRNA-LNP-F2) via intramuscular route were observed through IVIS 3D mouse live image to obtain the luminescence data. A high-intensity luminescence signal was detected 1 day after injection. The signal of mRNA-LNP-F2 is higher than mRNA-LNP-FM under low dose (0.2 μg and 2.0 μg), and the signal difference between the signals of mRNA-LNP-F2 and mRNA-LNP-FM are less obvious under high dose (10 μg). The signal showed a downward trend within one week of injection, and the luminescence signal could not be detected after that. It is speculated that the time for mRNA-LNP to express the vaccine antigen in the body is about one week.

### Example 6—PEGylated Lipid Optimization of mRNA-LNPs

The methods for preparing the nucleic acid-LNP are similar to those described in Example 1 and are not repeated again.

TABLE 3

| | Characteristics | | Molar ratio (mol %) of lipids | | | | Types of |
|---|---|---|---|---|---|---|---|
| | Size | EE | | | | PEGylated | PEGylated |
| Acronym | (nm) | (%) | SM-102 | Cholesterol | DSPC | lipid | lipid |
| mRNA-LNP-F2 | 135 | 80 | 50 | 38.5 | 10 | 1.5 | PLA 500- |
| | | | (50 mol %) | (38.5 mol %) | (10 mol %) | (1.5 mol %) | PEG 2000 |
| mRNA-LNP-F21 | 215 | 24 | 50 | 38.5 | 10 | 1.5 | PLA 500- |
| | | | (50 mol %) | (38.5 mol %) | (10 mol %) | (1.5 mol %) | PEG 4600- |
| | | | | | | | PLA500 |
| mRNA-LNP-F22 | 220 | 22 | 50 | 38.5 | 10 | 1.5 | PLA 250- |
| | | | (50 mol %) | (38.5 mol %) | (10 mol %) | (1.5 mol %) | PEG 4600- |
| | | | | | | | PLA250 |

The highly reproducible mRNA-lipid nanoparticle can be prepared by a microfluidic mixer (Precision Nanosystems, Vancouver, BC, Canada). By adjusting the structure of PEGylated lipid in LNP, the particle sizes of the lipid nanoparticles (LNP-F21 and LNP-F22) are increased (˜220 nm) and the encapsulation efficiency of mRNA are decreased (˜20%).

### Example 7—Preparation and Characterization of DNA-LNPs

DNA-LNP formulations were elaborated using microfluidic approaches. Briefly, lipids (ionizable lipid:cholesterol:helper lipid: PEGylated lipid) were dissolved in ethanol at predetermined DNA to lipids weight ratios. The lipid mixture was combined with a DNA-containing buffer solution at a volume ratio of 1/3 (ethanol/aqueous) using a microfluidic mixer (Precision Nanosystems, Vancouver, BC, Canada) as the same with mRNA-LNP formulations. Formulations were then concentrated and stored in the TRIS (pH 7.4). LNP characterization was performed, including particle size distribution using laser light scattering techniques, DNA determination and encapsulation efficiency (EE) using fluorescence detection method (RiboGreen™ RNA Reagent and Quantitation Kit). For DNA transfection, 293T cells were seeded in 24-well plates at 10⁵ cells/well and transfected with DNA-GFP (1.0 μg/ml) using DNA Turbojet Transfection Reagent or LNP-encapsulated. 72 hours after transfection, the cells were collected and monitored by optical microscope (OM).

TABLE 4

| | Characteristics | | | Molar ratio (mol %) of lipids (with fixed cholesterol content | | | |
|---|---|---|---|---|---|---|---|
| | EE | Size | DNA | of 516 μg) | | | |
| Acronym | (%) | (nm) | (μg) | SM-102 | Cholesterol | DSPC | DMG-PEG 2000 |
| DNA-LNP-FM | 96 | 115 | 100 | 50 | 38.5 | 10 | 1.5 |
| | | | | (50 mol %) | (38.5 mol %) | (10 mol %) | (1.5 mol %) |
| DNA-LNP-FM1 | 88 | 114 | 100 | 25 | 38.5 | 10 | 1.5 |
| | | | | (33 mol %) | (50 mol %) | (13 mol %) | (4 mol %) |
| DNA-LNP-FM2 | 40 | 111 | 200 | 50 | 38.5 | 10 | 1.5 |
| | | | | (50 mol %) | (38.5 mol %) | (10 mol %) | (1.5 mol %) |

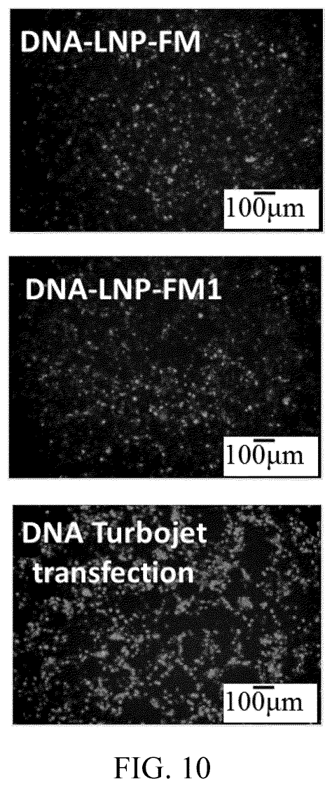

The results are shown in Table 4 and FIG. 10. The DNA with green fluorescent protein (GFP) sequence, under the formula DNA-LNP-FM, can be encapsulated within the lipid mixture to obtain a uniform DNA-lipid nanoparticle by using a microfluidic mixer (Precision Nanosystems, Vancouver, BC, Canada). The encapsulation efficiency was 96% measured by fluorescent dye detection, and its particle diameter detected by laser light scattering technology was 115 nm. If the ionizable lipid amount was decreased to half of DNA-LNP-FM (denoted as DNA-LNP-FM1), the encapsulation efficiency was slightly reduced (88%). If the DNA amount was increased to double of DNA-LNP-FM (denoted as DNA-LNP-FM2), the encapsulation efficiency was reduced to 40% o. The results of the in vitro cell experiments indicated that DNA-LNP-FMI can significantly improve the protein expression level of DNA with green fluorescent protein sequence in 293T cells.

### Example 8—Parameter Tuning for the Preparation of DNA-LNPs

The methods for preparing the DNA-LNPs are similar to those described in Example 7 and are not repeated again.

TABLE 5

| | | Molar ratio of lipids (with fixed | | | |
|---|---|---|---|---|---|
| | | cholesterol content of 516 μg) | | | |
| | DNA | | | | PLA |
| | content | | | | 500-PEG |
| Acronym | (μg) | Cholesterol | DSPC | SM-102 | 2000 |
| PLA-PEG-0 | 100 | 38.5 | 10.0 | 50 | 0.0 |
| PLA-PEG-0.5 | 100 | 38.5 | 10.0 | 50 | 0.5 |
| PLA-PEG-1.0 | 100 | 38.5 | 10.0 | 50 | 1.0 |
| PLA-PEG-1.5 | 100 | 38.5 | 10.0 | 50 | 1.5 |
| PLA-PEG-2.0 | 100 | 38.5 | 10.0 | 50 | 2.0 |
| PLA-PEG-3.0 | 100 | 38.5 | 10.0 | 50 | 3.0 |
| DSPC-0 | 100 | 38.5 | 0.0 | 50 | 1.5 |
| DSPC-5 | 100 | 38.5 | 5.0 | 50 | 1.5 |
| DSPC-10 | 100 | 38.5 | 10.0 | 50 | 1.5 |
| DSPC-20 | 100 | 38.5 | 20.0 | 50 | 1.5 |
| P/H-2.5/9 | 100 | 38.5 | 9.0 | 50 | 2.5 |
| P/H-2/9.5 | 100 | 38.5 | 9.5 | 50 | 2.0 |
| P/H-1.5/10 | 100 | 38.5 | 10.0 | 50 | 1.5 |
| P/H-1/10.5 | 100 | 38.5 | 10.5 | 50 | 1.0 |
| P/H-0.5/11 | 100 | 38.5 | 11.0 | 50 | 0.5 |
| SM-102-20 | 100 | 38.5 | 10.0 | 20 | 1.5 |
| SM-102-30 | 100 | 38.5 | 10.0 | 30 | 1.5 |
| SM-102-40 | 100 | 38.5 | 10.0 | 40 | 1.5 |
| SM-102-50 | 100 | 38.5 | 10.0 | 50 | 1.5 |
| SM-102-60 | 100 | 38.5 | 10.0 | 60 | 1.5 |
| SM-102-20-0 | 100 | 38.5 | 10.0 | 20 | 0.0 |
| DNA-50 | 50 | 38.5 | 10.0 | 50 | 1.5 |
| DNA-66 | 66 | 38.5 | 10.0 | 50 | 1.5 |
| DNA-100 | 100 | 38.5 | 10.0 | 50 | 1.5 |
| DNA-200 | 200 | 38.5 | 10.0 | 50 | 1.5 |

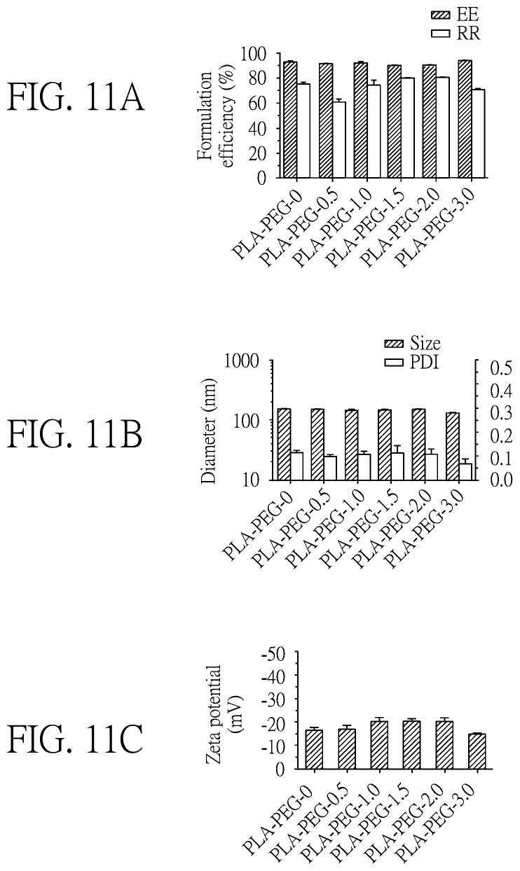

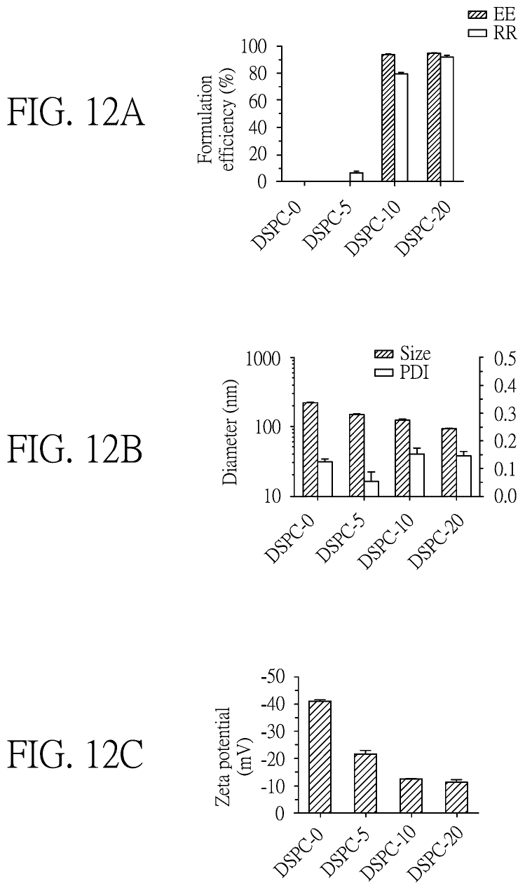

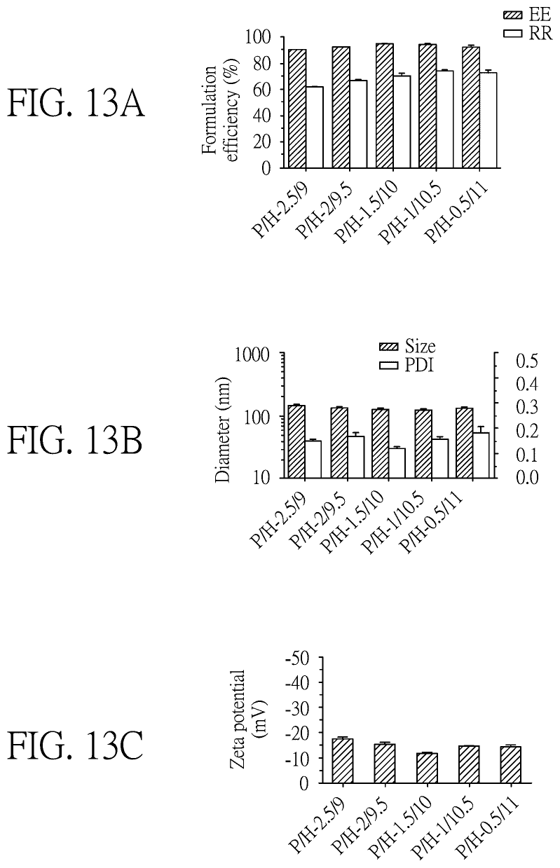

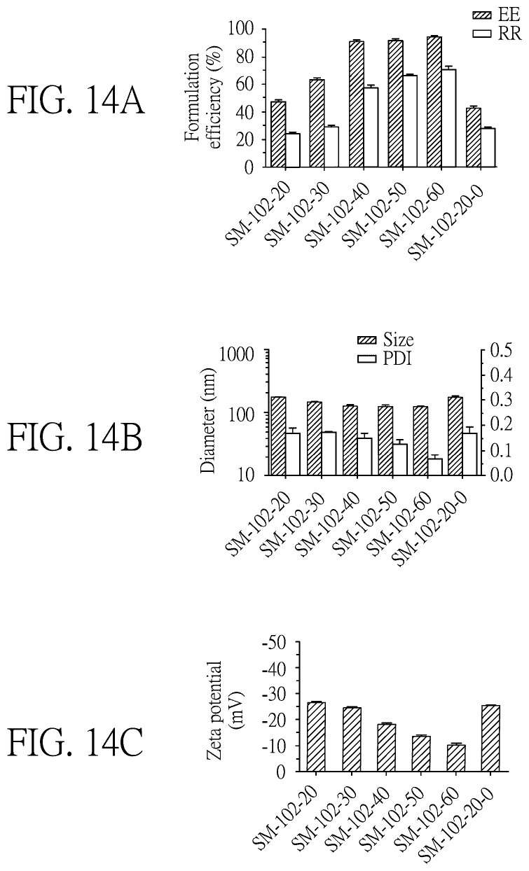

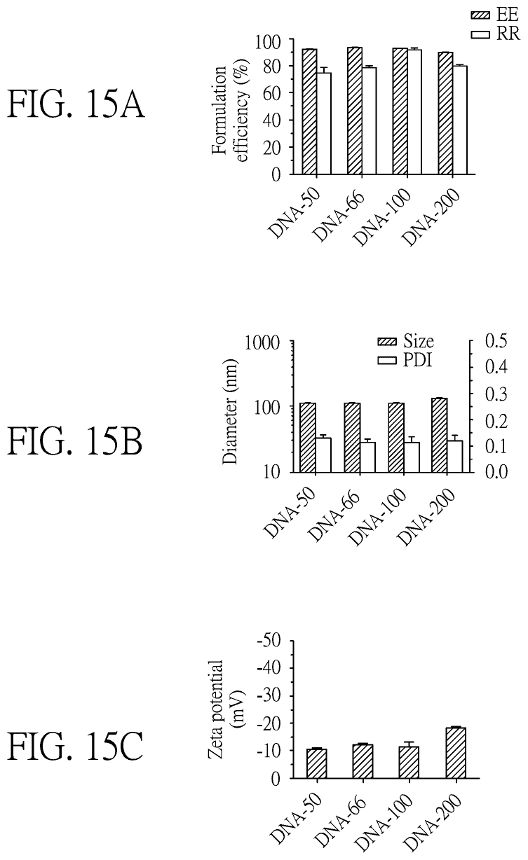

The microfluidic mixer (Precision Nanosystems, Vancouver, BC, Canada) was used to prepare DNA-LNPs, and the contents of the lipid mixture of the DNA-LNPs were adjusted, as shown in Table 5. As shown in FIG. 11A to FIG. 15C, it was found that PEGylated lipid (PLA-PEG), the ratio between PEGylated lipid and helper lipid, and the DNA content have little effect on the properties of lipid nanoparticles. If the content of the helper lipid (DSPC) is insufficient (<5 mol %), the DNA molecule cannot be encapsulated. In addition, the content of the ionizable cationic lipid (SM-102) also affects the encapsulation efficiency (EE) and recovery rate (RR) of DNA, as well as the particle size, the distribution and the surface charge.

### Example 9—Stability of DNA-LNPs

Stability monitoring of the DNA-LNP-F2 formulations was performed at room temperature and 4° C., 15° C., 25° C., 37° C. The methods for preparing the DNA-LNP-F2 formulations (lipids mixture with molar ratios of 50:38.5:10:1.5 (SM-102:cholesterol:DSPC:PLA500-PEG 2000); EE (%)=97%) are similar to those described in Example 7 and are not repeated again. At week 4, the specimens were withdrawn and characterized by particle size distribution using laser light scattering techniques, DNA determination and encapsulation efficiency (EE) using fluorescence detection method (RiboGreen™ NA Reagent and Quantitation Kit). For DNA transfection, 293T cells were seeded in 24-well plates at 10⁵ cells/well and transfected with DNA-luciferase (1.0 g/ml) in triplicate cultures for 48 hrs.

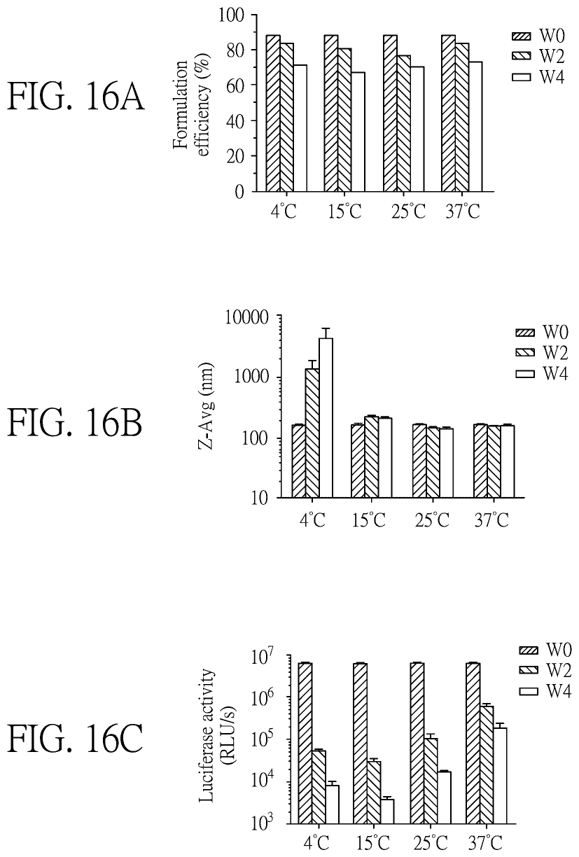

As shown in FIG. 16A to FIG. 16C, the luciferase-DNA-LNP-F2 formulations were stored at 4° C., 15° C., 25° C. and 37° C. for 4 weeks, and the stability and the protein expressions of the luciferase-DNA-LNP-F2 formulations were monitored. The diameters of the LNP formulations remained unchanged, and the luminescence signal produced by the cells decreased the least when storing at 37° C. However, the diameters of the LNP formulations increased, and the luminescent signal produced by cells decreases the most when storing at 4° C.

### Example 10—In Vivo Bioluminescence of DNA-LNPs

Female BALB/c mice were inoculated with 10 μg of DNA-LNP-F2 via intramuscular (i.m.) route. Day 1, 2, 3, 4, 7, 10, 14, 21, 28 after administrations, animals were injected intraperitoneally (i.p.) with luciferase substrate, and waited 11 minutes for reaction. Fluorescence signals were recorded by IVIS Spectrum instrument, and the fluorescence signals in regions of interest (ROIs) were quantified using Living Image.

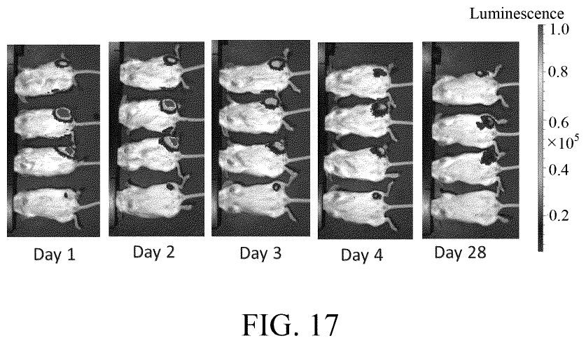

The mice treated with DNA-luciferase in the lipid mixture of the present invention (DNA-LNP-F2) via intramuscular route were observed through IVIS 3D mouse live image to obtain the luminescence data. The results are shown in FIG. 17. A high-intensity luminescence signal was detected 1 day after the injection of 10 μg of DNA-LNP-F2. The signal showed a downward trend within 4 days of injection, and the luminescence signal could still be detected within one month after injection.

### Example 11—PEGylated Lipid Optimization of DNA-LNPs

The methods for preparing the DNA-LNPs are similar to those described in Example 7 and are not repeated again. The amounts of the components in the DNA-LNPs are listed in the following Table 6.

TABLE 6

| | Molar ratio (mol %) of lipids | | | | Types of |
|---|---|---|---|---|---|
| | | | | PEGylated | PEGylated |
| Acronym | SM-102 | Cholesterol | DSPC | lipid | lipid |
| J006-0 | 50 | 38.5 | 10 | — | — |
| | (50 mol %) | (38.5 mol %) | (10 mol %) | | |
| J006-0.275 | 50 | 38.5 | 10 | 0.275 | PLACL-PEG^{a} |
| | (50.6 mol %) | (39.0 mol %) | (10.1 mol %) | (0.3 mol %) | |
| J006-0.55 | 50 | 38.5 | 10 | 0.55 | PLACL-PEG^{a} |
| | (50.5 mol %) | (38.9 mol %) | (10.1 mol %) | (0.6 mol %) | |
| J006-1.1 | 50 | 38.5 | 10 | 1.1 | PLACL-PEG^{a} |
| | (50.2 mol %) | (38.7 mol %) | (10.0 mol %) | (1.1 mol %) | |
| DMG-PEG-0.55 | 50 | 38.5 | 10 | 1.5 | DMG-PEG 2000 |
| | (50 mol %) | (38.5 mol %) | (10 mol %) | (1.5 mol %) | |
| Y001-0.55 | 50 | 38.5 | 10 | 1.5 | PLA 500-PEG 2000 |
| | (50 mol %) | (38.5 mol %) | (10 mol %) | (1.5 mol %) | |

^{a}PLACL-PEG denotes poly(lactide-co-ε-caprolactone)-block-poly(ethylene glycol) with average molecular weight of poly(lactide-co-ε-caprolactone) being 2,000 Daltons; and average molecular weight of PEG being 5,000 Daltons.

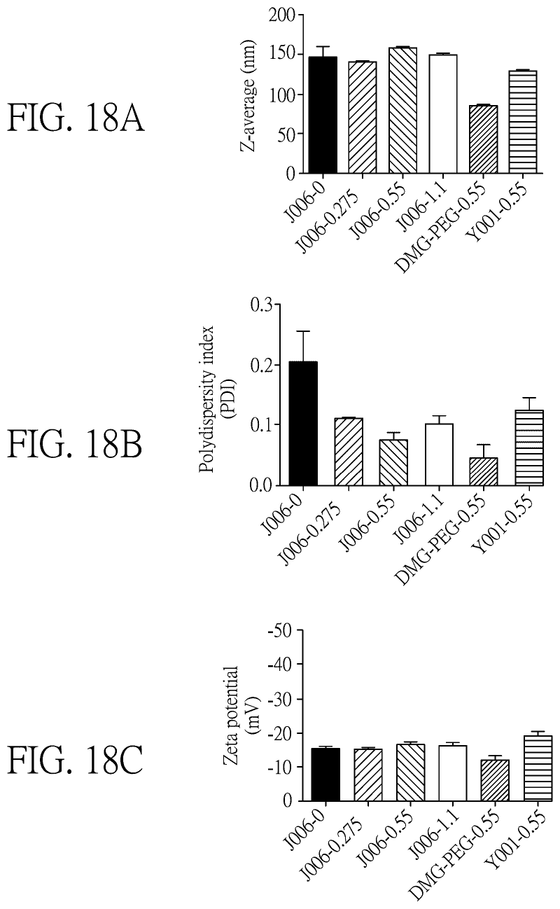

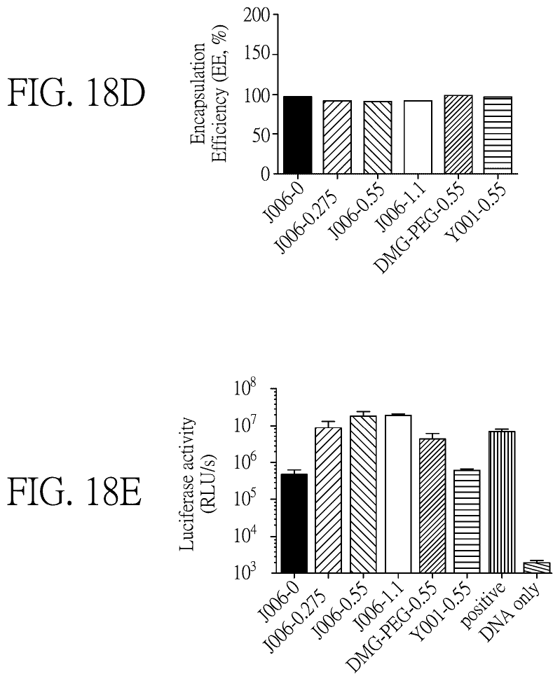

As shown in FIG. 18A to FIG. 18E, in the formulation of DMG-PEG-0.55, a uniform and solid nanoparticle can be prepared by a microfluidic mixer (Precision Nanosystems, Vancouver, BC, Canada). When the PEGylated lipid in the LNP was removed, the lipid nanoparticles (J006-0) were easily aggregated, the distribution (PDI) of lipid nanoparticles was increased, and the protein expression of the DNA molecule was decreased. Supplements of appropriate amounts of J006 were facilitated to form DNA-LNP particles with high protein expression and stability. The data of the present example also compare PEGylated derivatives with different molecular weight ratios and hydrolytic properties. It was found that increasing the length of the polyethylene glycol segment can improve the protein expression effect of nucleic acid sequences in cells, showing that this parameter is very important for nucleic acid vaccine formulations.

### Example 12—Ionizable Cationic Lipid Optimization of DNA-LNPs

The methods for preparing the DNA-LNPs are similar to those described in Example 7 and are not repeated again. The amounts of the components in the DNA-LNPs are listed in the following Table 7.

TABLE 7

| | Molar ratio (mol %) of lipids | | | | Types of | Types of |
|---|---|---|---|---|---|---|
| | Ionizable | | | PEGylated | PEGylated | Ionizable |
| Acronym | cationic lipid | Cholesterol | DSPC | lipid | lipid | cationic lipid |
| PLA-PEG/ | 46.3 | 42.7 | 9.4 | 1.6 | PLA 500- | ACL-0315 |
| ACL0315 | (46.3 mol %) | (42.7 mol %) | (9.4 mol %) | (1.6 mol %) | PEG 2000 | |
| PLA-PEG/ | 50 | 38.5 | 10 | 1.5 | PLA 500- | DLin-MC3-DMA |
| MC3 | (50 mol %) | (38.5 mol %) | (10 mol %) | (1.5 mol %) | PEG 2000 | |
| PLA-PEG/ | 50 | 38.5 | 10 | 1.5 | PLA 500- | SM102 |
| SM102 | (50 mol %) | (38.5 mol %) | (10 mol %) | (1.5 mol %) | PEG 2000 | |
| DMG-PEG/ | 50 | 38.5 | 10 | 1.5 | DMG-PEG | SM102 |
| SM102 | (50 mol %) | (38.5 mol %) | (10 mol %) | (1.5 mol %) | 2000 | |

In the present example, for DNA transfection, RAW264.7 cells were also used in the DNA transfection, and the method was similar to that using 293T cells and is not repeated again.

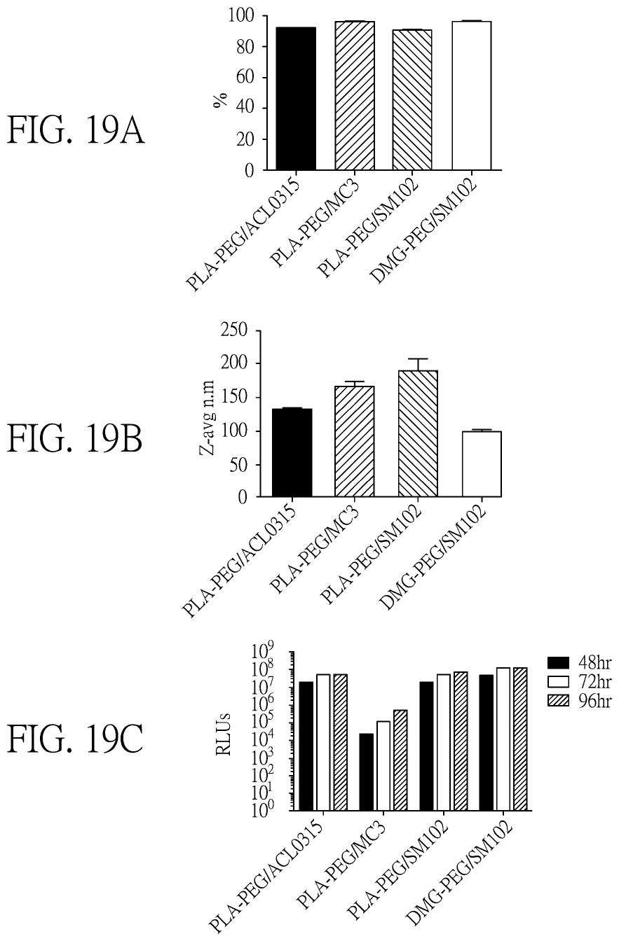

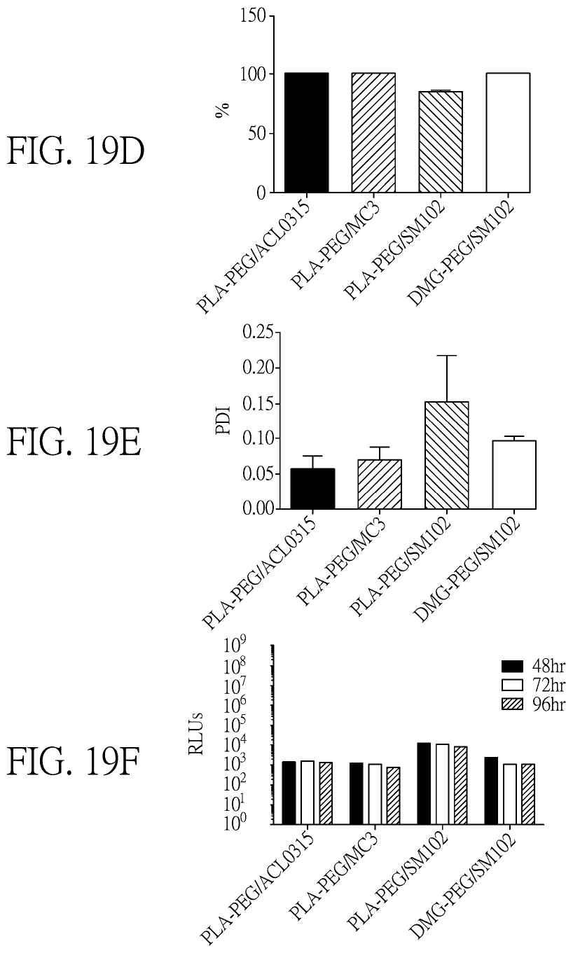

The PEGylated lipid used in the LNPs disclosed by Modena, Pfizer/BNT and Alnylam Company is replaced by the PLA-PEG of the present invention. The particle size, encapsulation efficiency and recovery rate of DNA, and protein expression were evaluated, and the results are shown in FIG. 19A to FIG. 19F. It is found that there is little difference between the encapsulation efficiency and the recovery rate. When the PLA-PEG of the present invention is used with SM102, LNP has the largest particle size range, and the cells produce the most luminescence signals.

### Example 13—Immunogenicity of Nucleic Acid Encoding Trimeric Spike Proteins in Hamsters

The methods for preparing the nucleic acid-LNPs are similar to those described in Examples 1 and 7 and are not repeated again. The amounts of the components in the nucleic acid-LNPs are listed in the following Table 8.

TABLE 8

| | | Molar ratio (mol %) of lipids | | | | |
|---|---|---|---|---|---|---|
| | | | | | PEGylated | Types of |
| Acronym | EE (%) | SM-102 | Cholesterol | DSPC | lipid | PEGylated lipid |
| mRNA-LNP-FM | 83 | 50 | 38.5 | 10 | 1.5 | DMG-PEG 2000 |
| | | (50 mol %) | (38.5 mol %) | (10 mol %) | (1.5 mol %) | |
| DNA-LNP-FM | 95 | 50 | 38.5 | 10 | 1.5 | DMG-PEG 2000 |
| | | (50 mol %) | (38.5 mol %) | (10 mol %) | (1.5 mol %) | |
| DNA-LNP-F2 | 97 | 50 | 38.5 | 10 | 1.5 | PLA 500-PEG 2000 |
| | | (50 mol %) | (38.5 mol %) | (10 mol %) | (1.5 mol %) | |
| mRNA-LNP-F2 | 94 | 50 | 38.5 | 10 | 1.5 | PLA 500-PEG 2000 |
| | | (50 mol %) | (38.5 mol %) | (10 mol %) | (1.5 mol %) | |

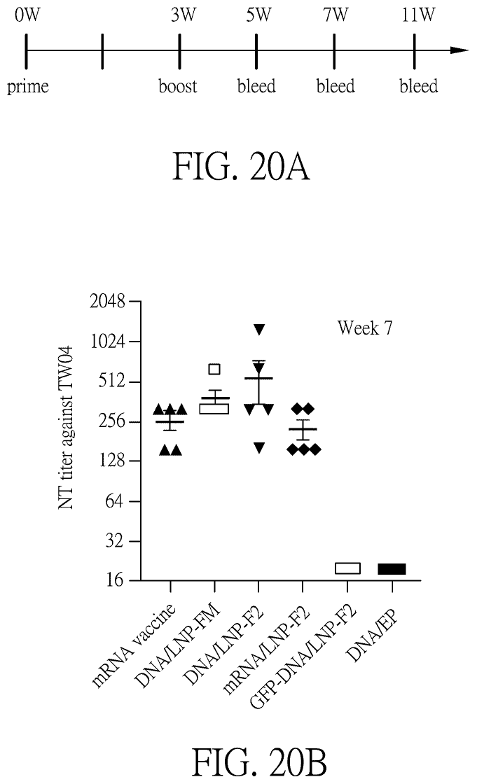

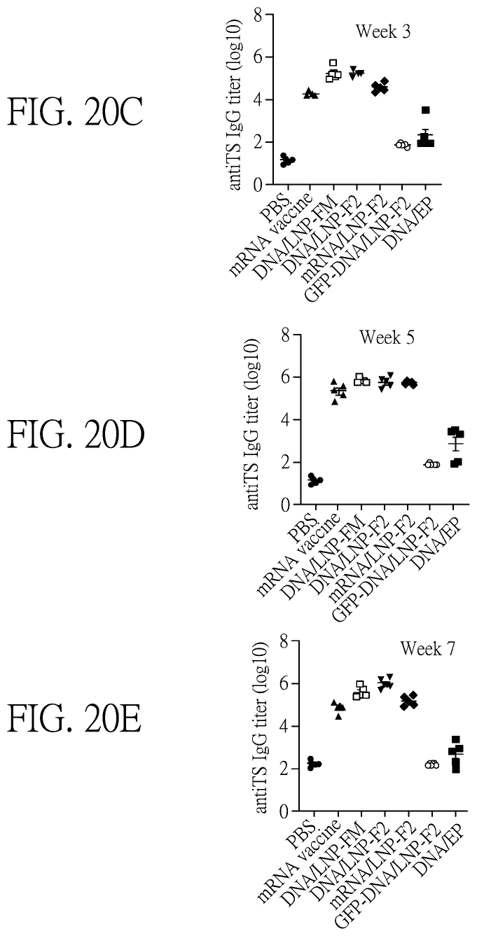

FIG. 20A is a schematic diagram of vaccination schedule. Hamsters were immunized with 10 μg DNA and mRNA vaccines at Day 0 and week 3. The week 5, 7 and 11 sera were against TW04 (wuhan strain) strain to evaluate neutralizing antibody titer, and the results are shown in FIG. 20B. In addition, the IgG antibodies in serum against trimeric spike protein (TS) are expressed as the individual values with the GMT, which were obtained by enzyme-linked immunosorbent assay (ELISA), and the results are shown in FIG. 20C to FIG. 20E.

The results of FIG. 20B to FIG. 20E indicate that the DNA-LNP vaccine (DNA-LNP-FM and DNA-LNP-F2) can induce high titer neutralizing virus antibody without using an external electric field. However, the DNA molecules alone cannot induce such high titer neutralizing virus antibody even using an external electric field (DNA/EP). In addition, DNA vaccine immunization (DNA-LNP-FM and DNA-LNP-F2) induced antigen-specific antibodies is better than the same dose of mRNA vaccine (mRNA vaccine and mRNA-LNP-F2).

### Example 14—Immunogenicity of Nucleic Acid Encoding Trimeric Spike Proteins in Hamsters

The methods for preparing the nucleic acid-LNPs are similar to those described in Examples 1 and 7 and are not repeated again. The amounts of the components in the nucleic acid-LNPs are listed in the following Table 9.

TABLE 9

| | mRNA or | Molar ratio (mol %) of lipids | | | | Types of |
|---|---|---|---|---|---|---|
| | DNA content | | | | PEGylated | PEGylated |
| Acronym | (μg) | SM-102 | Cholesterol | DSPC | lipid | lipid |
| Tris buffer | 0 | — | — | — | — | — |
| mRNA-LNP-FM | mRNA, 10 μg | 50 | 38.5 | 10 | 1.5 | DMG- |
| | | (50 mol %) | (38.5 mol %) | (10 mol %) | (1.5 mol %) | PEG 2000 |
| DNA-LNP-F2 | DNA, 1 μg | 50 | 38.5 | 10 | 1.5 | PLA 500- |
| | | (50 mol %) | (38.5 mol %) | (10 mol %) | (1.5 mol %) | PEG 2000 |
| DNA-LNP-F2 | DNA, 10 μg | 50 | 38.5 | 10 | 1.5 | PLA 500- |
| | | (50 mol %) | (38.5 mol %) | (10 mol %) | (1.5 mol %) | PEG 2000 |
| DNA-LNP-F2 | DNA, 30 μg | 50 | 38.5 | 10 | 1.5 | PLA 500- |
| | | (50 mol %) | (38.5 mol %) | (10 mol %) | (1.5 mol %) | PEG 2000 |

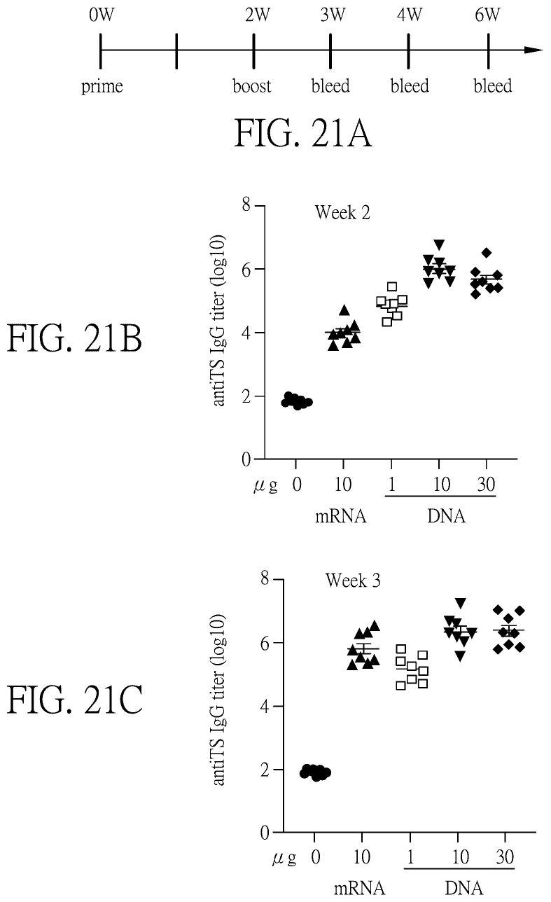

FIG. 21A is a schematic diagram of vaccination schedule. Hamsters were immunized with DNA and mRNA vaccines at Day 0 and week 2. The IgG antibodies in serum against trimeric spike protein (TS) are expressed as the individual values with the GMT, which were obtained by enzyme-linked immunosorbent assay (ELISA), and the results are shown in FIG. 21B to FIG. 21C.

The results of FIG. 21B to FIG. 21C indicate that the DNA vaccine immunization (DNA-LNP-F2) induced antigen-specific antibodies is better than the mRNA vaccine (mRNA-LNP-FM).

### Example 15—Vaccine Efficacy of LNP-Formulated DNA Sequences Encoding SARS-CoV-2 Omicron Variant Spike Genes Against Wuhan Original Strain

The methods for preparing the DNA-LNPs are similar to those described in Example 7 and are not repeated again. The amounts of the components in the DNA-LNPs are listed in the following Table 10.

TABLE 10

| | | Molar ratio (mol %) of lipids | | | | Types of |
|---|---|---|---|---|---|---|
| | DNA content | | | | PEGylated | PEGylated |
| Acronym | (μg) | SM-102 | Cholesterol | DSPC | lipid | lipid |
| DNA-LNP-10 | DNA, 10 μg | 50 | 38.5 | 10 | 1.5 | PLA 500- |
| μg | | (50 mol %) | (38.5 mol %) | (10 mol %) | (1.5 mol %) | PEG 2000 |
| DNA-LNP-30 | DNA, 30 μg | 50 | 38.5 | 10 | 1.5 | PLA 500- |
| μg | | (50 mol %) | (38.5 mol %) | (10 mol %) | (1.5 mol %) | PEG 2000 |

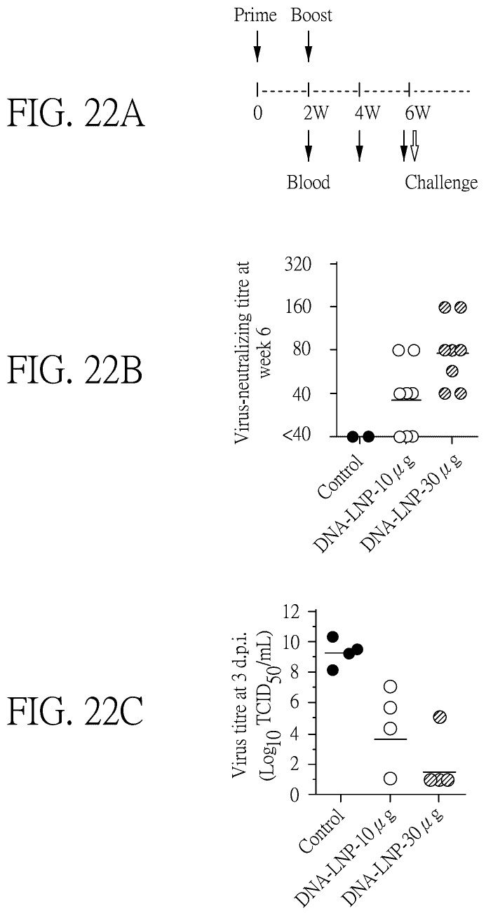

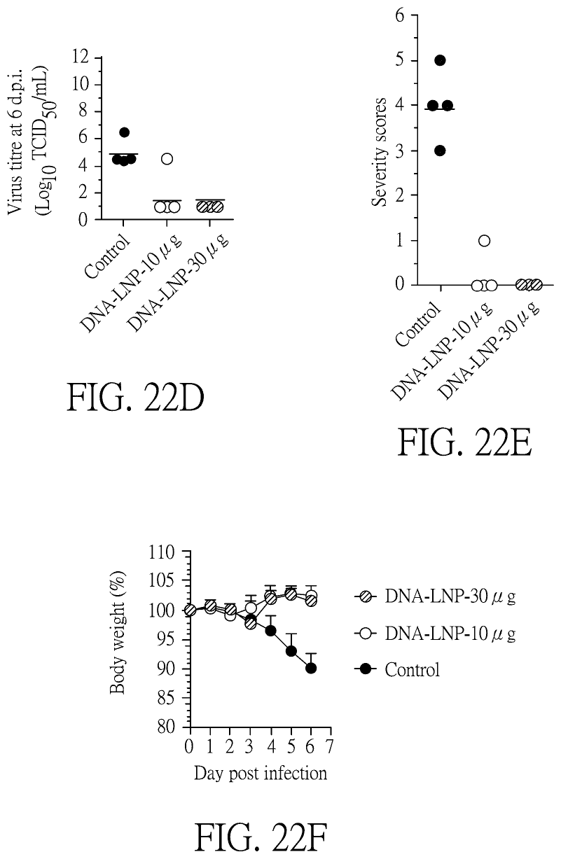

As shown in FIG. 22A, Hamsters (n=8) were intramuscularly (i.m.) injected with DNA-LNP formulations on day 0. At day 14, all mice were boosted i.m. with the same vaccine formulations. In addition, the hamsters were challenged with Wuhan SARS-CoV-2 at day 42 post vaccination.

FIG. 22B shows the virus neutralizing (VN) antibodies in serum samples at day 42 are expressed as the individual values with the GMT. The log-transformed values of antibody titres were compared by performing one-way ANOVA model followed by Dunnet's multiple comparison test. FIG. 22C and FIG. 22D show the viral load in the lungs of infected hamsters at 3- and 6-days post-infection (d.p.i.) determined by TCID₅₀ assay. FIG. 22E shows the histopathological examination of tissue sections (4 μm) at the lungs of infected hamsters which were performed by staining with haematoxylin and eosin (H&E) and examined using a microscope (original magnification, ×40 and ×400). Pathologic severity was scored and the p value was calculated by t test. FIG. 22F shows a diagram of the body weight change (%) after virus challenge. The above results indicate that the DNA-LNP formulations of the present invention can effectively induce antigen-specific antibodies.

### Example 16—Boost Effect Pf DNA-LNP Formulations

Hamsters (n=5) were intramuscularly (i.m.) injected with 10 μg Wuhan mRNA-LNP-FM on day 0 and day 21. At week 26, the hamsters were boosted i.m. with 10 μg Omicron DNA-LNP-F2. The Omicron VN antibodies in serum samples before and two weeks after the boost are expressed as the individual values with the GMT. The log-transformed values of antibody titres were compared by performing ANOVA model followed by Dunnet's multiple comparison test. The dotted horizontal line represents the seroconversion.

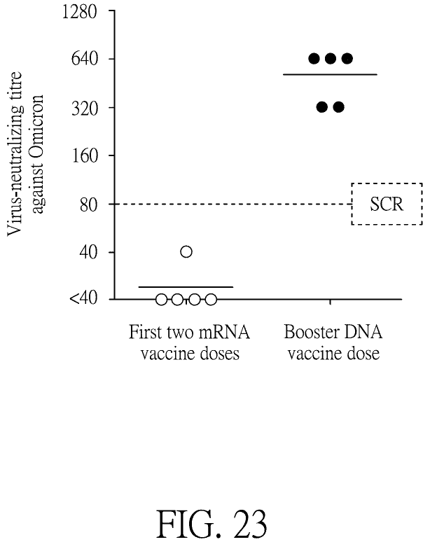

The results shown in FIG. 23 indicate that the DNA-LNP formulation can be used as a booster dose against SARS-CoV-2 Omicron variant.

Although the present disclosure has been explained in relation to its embodiment, it is to be understood that many other possible modifications and variations can be made without departing from the spirit and scope of the disclosure as hereinafter claimed.

1-20. (canceled)

21. A nucleic acid-lipid nanoparticle, comprising:
a nucleic acid molecule; and
a lipid mixture comprising:
an ionizable amino lipid present in an amount of 20 mol % to 60 mol %;
a phospholipid present in an amount of 5 mol % to 20 mol %;
cholesterol present in an amount of 25 mol % to 60 mol %; and
a PEGylated lipid present in an amount of 0.2 mol % to 6 mol %, wherein the PEGylated lipid comprises a copolymer having a first block of PLA and a second block of PEG.

22. A nucleic acid-lipid nanoparticle, comprising:
a nucleic acid molecule; and
a lipid mixture comprising:
an ionizable amino lipid present in an amount of 20 mol % to 60 mol %;
a phospholipid present in an amount of 5 mol % to 20 mol %;
cholesterol present in an amount of 25 mol % to 60 mol %; and
a PEGylated lipid present in an amount of 0.2 mol % to 6 mol %, wherein the PEGylated lipid comprises a copolymer having a first block of poly(lactide-co-F-caprolactone) and a second block of PEG.

23. The nucleic acid-lipid nanoparticle of claim 21 or 22, wherein the nucleic acid molecule comprises a messenger RNA (mRNA) or a DNA encoding a part of a coronavirus.

24. The nucleic acid-lipid nanoparticle of claim 21 or 22, wherein the nucleic acid molecule comprises a mRNA or a DNA encoding a trimeric spike protein of SARS-CoV-2 virus.

25. The nucleic acid-lipid nanoparticle of claim 21 or 22, wherein the ionizable amino lipid is selected from the group consisting of heptadecan-9-yl 8-((2-hydroxyethyl) (6-oxo-6-(undecyloxy) hexyl)amino)octanoate (SM-102), (6Z,9Z,28Z,31Z)-heptatriaconta-6,9,28,31-tetraen-19-yl-4-(dimethylamino)butanoate (DLin-MC3-DMA), ((4-hydroxybutyl)azanediyl)bis(hexane-6,1-diyl)bis(2-hexyldecanoate) (ALC-0315) and a combination thereof.

26. The nucleic acid-lipid nanoparticle of claim 21 or 22, wherein the phospholipid is selected from the group consisting of dioleoylphosphatidyl ethanolamine (DOPE), dimyristoylphosphatidyl choline (DMPC), 1,2-distearoyl-sn-glycero-3-phosphocholine (DSPC), dimyristoylphosphatidyl glycerol (DMPG), dipalmitoyl phosphatidylcholine (DPPC), phosphatidylcholine (PC) and a combination thereof.

27. The nucleic acid-lipid nanoparticle of claim 21, wherein the first block of PLA has an average molecular weight of 200 Daltons to 600 Daltons.

28. The nucleic acid-lipid nanoparticle of claim 21, wherein the second block of PEG has an average molecular weight of 1,500 Daltons to 6,000 Daltons.

29. The nucleic acid-lipid nanoparticle of claim 21, wherein the copolymer further has a third block of PLA, and the second block of PEG is arranged between the first block of PLA and the third block of PLA.

30. The nucleic acid-lipid nanoparticle of claim 29, wherein the third block of PLA has an average molecular weight of 200 Daltons to 600 Daltons.

31. The nucleic acid-lipid nanoparticle of claim 22 wherein the second block of PEG has an average molecular weight of 4,000 Daltons to 6,000 Daltons.

32. The nucleic acid-lipid nanoparticle of claim 22, wherein the first block of poly(lactide-co-ε-caprolactone) has an average molecular weight of 1,500 Daltons to 2,500 Daltons.

33. The nucleic acid-lipid nanoparticle of claim 21 or 22, wherein a weight ratio of the lipid mixture to the nucleic acid molecule ranges from 50:1 to 5:1.

34. The nucleic acid-lipid nanoparticle of claim 21 or 22, wherein the nucleic acid-lipid nanoparticle has a median diameter ranging from 80 nm to 240 nm.

35. A method for preventing or ameliorating a coronavirus infection, comprising administering the nucleic acid-lipid nanoparticle of claim 21 or 22 to a subject in need thereof.

36. A method for inducing an immune response against a coronavirus in a subject, comprising administering the nucleic acid-lipid nanoparticle of claim 21 or 22 to a subject in need thereof.

37. A method for administering a booster dose to a vaccinated subject, comprising administering the nucleic acid-lipid nanoparticle of claim 21 or 22 to a subject who was previously vaccinated against coronavirus.
